(19) United States
(12) Patent Application Publication
(10) Pub. No.: US 2006/0154256 A1
(43) Pub. Date: Jul. 13, 2006

(54) Method for covalently attaching nucleosides and/or nucleotides on surfaces and method for determining coupling yields in the synthesis of nucleotides

(76) Inventors: Klaus-Peter Stengele, Pleiskirchen (DE); Evgueni Kvassiouk, Waldkraiburg (DE)

Correspondence Address: GRAYBEAL, JACKSON, HALEY LLP, 155 - 108TH AVENUE NE, SUITE 350, BELLEVUE, WA, 98004-5901, US

(21) Appl. No.: 10/831,532

(22) Filed: Apr. 23, 2004

(30) Foreign Application Priority Data
Oct. 25, 2001 (DE) 101 52 147.2
Oct. 25, 2002 (WO) PCT/EP02/11938

(51) Int. Cl. C12Q 1/68; C07H 21/04

(52) U.S. Cl. 435/6; 536/25.32

(57) ABSTRACT

The present invention relates to a method for covalently attaching nucleosides and/or nucleotides on surfaces having reactive functional groups, where in a first step, the reactive functional groups are made to react with suitable derivatized nucleosides and/or nucleotides, and in a second step, they are converted with a protecting group reagent, so that a reaction product of the consecutive reaction interacts with electromagnetic radiation such that it can be quantitatively determined. The invention also relates to a method for determining the repetitive coupling yields in the synthesis of nucleotides where the free 3′ or 5′ hydroxy group of a selected nucleoside and/or nucleotide is converted with a compound of formula (I) 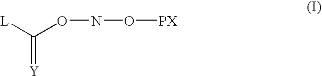
where L is a common suitable leaving group, the motif O—PX represents a phosphor amidite, a H-phosphonate a phosphonic acid ester, a phosphotriester, Y═O or S, N is a nucleoside or a nucleotide derivative which subsequently reacts further with a protecting group reagent and the elimination of the leaving group (L), which is subsequently further eliminated. The quantity of the leaving group (L) eliminated in step b) is quantitatively determined in the form of its anion (L⁻) by means of optical spectroscopy.

The present invention relates to a method for covalently attaching nucleosides and/or nucleotides on surfaces that have reactive functional groups, and a method for determining coupling yields in the synthesis of nucleotides.

The invention also relates to a kit for performing the methods of the invention.

In addition, the invention relates to the use of the methods or kits of the invention for producing nucleotides and/or nucleic acid chips.

The invention also relates to nucleoside derivatives and their use for the method of the invention.

Synthetic nucleotides are widely used in all areas of biotechnology and genetic engineering, such as in gene transfection or gene analysis. Nucleotides, which, means in the present context both oligonucleotides and polynucleotides, are produced by means of chain extension of a starting compound with many separate nucleoside structural elements. For the synthesis, the hydroxy groups of the starting compounds are derivatized such that a phosphodiester group or a phosphotriester group is formed during conversion. Other functional groups of the starting compounds which interfere with the conversion are blocked with commonly used protecting groups.

Further, DNA chips can be produced using 3′-O-phosphor amidites containing temporary photolabile protecting groups in the 5′-O position, (WO-A-96/18634). DE 199 15 867 A1 further describes photolabile protecting groups for hydroxy groups, where, in contrast with the above described method, the photolabile protecting group is introduced in the 3′-O position, so that the oligomers formed via a light-controlled synthesis are coupled to a solid phase via the 5′ end instead of the 3′ end, thereby allowing an enzyme reaction at the 3′ or 5′ end.

To make the method more efficient, nucleotides, especially polynucleotides, are usually synthesized via repetitive solid phase synthesis. The starting compounds are bound directly or via so-called linker groups to functionalized solid surfaces of polymer pellets or glass, metal or plastic surfaces and converted with the reagents required for a polynucleotide chain extension. Excess reagents and soluble reaction byproducts and solvents are easily removed from the solid phase-bound polynucleotide compounds.

The disadvantage of these methods in prior art is that because of the many separate reaction steps a low total yield is obtained, even if the individual yields are high. Therefore, depending on the desired nucleotide chain length and the number of separate reaction steps, a person skilled in the art has to use an considerable excess amount of starting compounds and reagents, which are very difficult to recover, if at all, after the conversion in order to be reused. In addition, the many undesired byproducts, which are often very similar to the final compound, have to be separated from the final product. Therefore, the person skilled in the art has to use a large quantity of the starting compounds and the products have to be purified at great expense. The latter problem was solved by providing a new method as disclosed in German patent application, file no. 10132536.3, which has not been published to date.

A common characteristic of the aforementioned methods is that previously it was very difficult, if not impossible, to determine the coupling yields in oligonucleotide synthesis, especially until the chain extension was completed. This was especially disadvantageous if the synthesis was performed directly on the surface of solid phase substrates because in the event of a faulty coupling a timely intervention was not possible. Therefore expensive reagents and instrument time were wasted. Furthermore, an accurate determination of where a faulty coupling had occured was previously only indirectly possible.

Although in principle it should be possible to determine repetitive coupling yields during photolytic, for example light induced synthesis by e.g. spectrophotometric determination of the released photolabile group, in practice all these approaches failed in so far as the released photoproduct itself is prone to further photoreactions. Therefore, it was impossible to obtain quantitative results. In addtition, the reaction mechanism of the photochemical reaction does not lead to a single but to a variety of different products with different physical and chemical properties.

Therefore, the object of the present invention is to provide a method that eliminates the above mentioned disadvantage of the prior art. In particular, such a method should be suitable for an automated solid phase synthesis of polynucleotides by repetitive coupling cycles where the coupling yields and thus the efficiency of the synthesis can easily be determined for each separate coupling cycle.

The problem underlying the invention is solved by a method for covalently attaching nucleosides and/or nucleotides on surfaces having reactive functional groups, comprising the following steps: Reaction of reactive functional groups with suitable derivatized nucleosides and/or nucleotides, whereby one hydroxyl group of the suitable derivatized nucleosides and/or nucleotides is protected with a first intermediary protecting group comprising a leaving group, reaction of the reaction product of step a) with a protecting group reagent suitable for forming a second intermediary protecting group whereby the leaving group is substituted, optionally quantitatively determining the free leaving group by its interaction with electromagnetic radiation.

The problem is further solved in that a new nucleotide derivative is provided especially for use in the method of the invention.

The term “covalently attaching” means in the context of the present invention that a covalent bond is formed between the functionalized or nonfunctionalized surface of a suitable substrate and nucleoside and/or nucleotide including polynucleotides.

The result of the interaction between a reaction product from the above mentioned stepwise reaction and electromagnetic radiation is that due to the numerous modern analytical methods that are currently available, such as nuclear magnetic resonance, UV/VIS, fluorescence spectroscopy, etc., which can easily be automated and parallelized, the coupling yield is easily, quickly and efficiently determined step-by-step by such a method.

Preferably, the reactive functional groups are substantially hydroxy groups as these are highly reactive and are able to react especially easily with the nucleosides and nucleotides to be applied.

According to a preferred embodiment the free hydroxy groups are reacted with a compound of the general formula 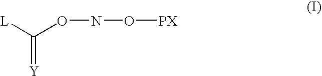
where L is a common suitable leaving group, such as electron-deficient substituted phenol or thiophenol derivatives, substituted and non-substituted polynuclear aromatic compounds with at least one hydroxy or thiol group, hetero-aromatic compounds, especially cyano and nitro derivatives of the above mentioned compounds, such as nitronaphthols, 4-nitrophenyloxy derivatives, etc. Further examples for L include but are not limited to 2,4-dinitrophenyloxy, pentafluorophenyloxy, phthalimideoxy, succinimideoxy and benzotriazolyloxy and the like.

The O—PX represents a phosphor amidite, H-phosphonate, a phosphonic acid ester, or a phosphotriester group. Phosphor amidites, H-phosphonates, phosphonic acid esters and phosphotriester useful in the context of the present invention are well known in the art and for example exhaustively reviewed by M. J. Gait “Oligonucleotide Synthesis—A practical approach”, IRL Press, 1984.

Y═O or S and N is a nucleoside or nucleotide fragment selected from:
a nucleoside fragment of formula (II), 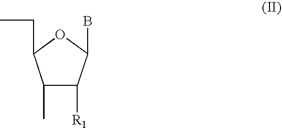
or a nucleotide fragment of formula (III) 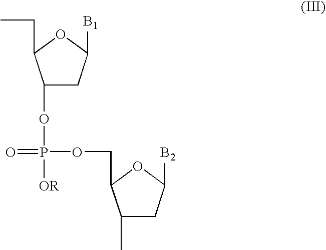
where the following applies: if in the respective terminal nucleoside, the PX-group is in the 3′ position in formulae (II) and (III), then the L-C(═Y) unit is in the 5′ position, and if the PX-group is in the 5′ position, then the L-C(═Y) unit is in the 3′ position,
and where B, B₁, B₂ independently can be H, adeninyl, cytosinyl, guaninyl, thyminyl, uracilyl, 2,6-diaminopurine-9-yl, hypoxanthine-9-yl, 5-methylcytosine-1-yl, 5-amino-4-imidazole carboxylic acid-1-yl or 5-amino-4-imidazole carboxylic acid amide-3-yl, where any primary amino functions that may be present in the case of B, B₁, B₂ could have a permanent protecting group, or thyminyl or uracilyl in the O₄ position could have a permanent protecting group,

R═H, an alkyl, aryl, aralkyl, haloalkyl, cyanoalkyl, and n=0 or an integer between 1 and 4, and if R═H, the compound is preferably present in the form of a soluble phosphor diester salt, for example in the form of a quaternary ammonium salt, and where R₁ can be an H, OH, halogen, acylamino, alkoxy or substituted alkoxy with between 1 and 4 C-atoms, or it can form a bicyclic compound via C2′-C4′ cyclization with the ribose unit (LNA locked nucleic acid).

Compound (I) especially if present as a phosphor amidite is obtained in excellent yields (up to 95%) and is very shelf-stable. This finding is quite surprising, because the synthesis, but also the use of compound (I) involves the use of activating additives as dicyanonimidazole, tetrazole, disisopropylammonium salts or pyridimium salts and the like. These additives dispose of a strong nucleophilic substitution potential, for example in oxidation reactions, when N-methylimidazole with water and a base like pyridine is used. A person skilled in the art would therefore expect, that compound (I) is not stable or that then entire first intermediate protecting group L-(C═Y)— is cleaved. The stability of compound (I) when exposed to the hitherto well known nucleophilic substitution agents as mentioned in the foregoing is unexpected.

The surprising finding of the present invention is therefore the selective cleavage of the leaving group L when compound (I) alone or already reacted with free reactive groups, expecially hydroxy groups is reacted with an alcohol preferably under DMAP catalysis conditions.

Preferably the leaving group L is eliminated in the second reaction step b) via a nucleophilic substitution reaction, more preferably via a catalytic nucleophilic substitution reaction, and can subsequently quantitatively be determined so that the coupling yield and efficiency (=yield) of the above method of the invention can be accurately determined.

For the method of the invention, pentanucleotides, especially dinucleotides, trinucleotides and tetranucleotides are preferably used for the selected oligonucleotides.

It is particularly preferred that the method of the invention comprises the following steps:
a) Reaction of a surface having a free hydroxy group with a compound with the general formula (I), 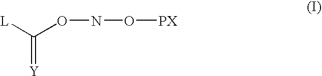
   where L is a common suitable leaving group, such as electron-deficient substituted phenol or thiophenol derivatives, substituted and non-substituted polynuclear aromatic compounds with at least one hydroxy or thiol group, hetero-aromatic compounds, etc., especially cyano and nitro derivatives of the above mentioned compounds, such as nitronaphthols, 4-nitrophenyloxy derivatives, etc. Further details with respect to L are described in the foregoing.

The O—PX motif represents a phosphor amidite, H-phosphonate, a phosphonic acid ester, or a phosphotriester group and examples of typical representatives of such compounds are given above.

Y means O or S, and N is a nucleoside or nucleotide fragment selected from:

a nucleoside fragment of formula (II), 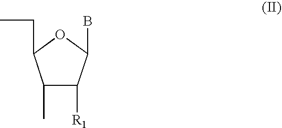
or a nucleotide fragment of formula (III) 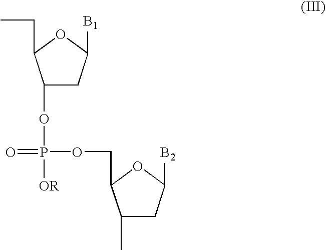
where the following applies: if in the respective terminal nucleoside, the PX-group is in the 3′ position in formulas (II) and (III), then the L-C(═Y) unit is in the 5′ position, and if the PX-group is in the 5′ position, then the L-C(═Y) unit is in the 3′ position,
and where B, B₁, B₂ independently can be H, adeninyl, cytosinyl, guaninyl, thyminyl, uracilyl, 2,6-diaminopurine-9-yl, hypoxanthine-9-yl, 5-methylcytosine-1-yl, 5-amino-4-imidazole carboxylic acid-1-yl or 5-amino-4-imidazole carboxylic acid amide-3-yl, where any primary amino functions that may be present in the case of B, B₁, B₂, could have a permanent protecting group, or thyminyl or uracilyl in the O₄ position could have a permanent protecting group,

R═H, an alkyl, aryl, aralkyl, haloalkyl, cyanoalkyl, and n=0 or an integer between 1 and 4, and if R═H, the compound is preferably present in the form of a soluble phosphor diester salt, for example in the form of a quaternary ammonium salt,

and where R₁ can be an H, OH, halogen, acylamino, alkoxy or substituted alkoxy with between 1 and 4 C-atoms, or it can form a bicyclic compound via C2′-C4′ cyclization with the ribose unit (LNA)

b) further reaction of the reaction product obtained in step a) which is preferably not photolabile with a second protecting group reagent suitable for the formation of a second protecting group, which is preferably a photolabile protecting group, and simultaneous elimination (substitution) of the leaving group L,
c) cleavage of the second protecting group introduced in step b), preferably with light (photolytic dissociation),
d) if required, repeating steps a) to c), using the reaction product obtained in step c) as the surface, and whereby the quantitative determination of the amount of the reaction product L, which was cleaved in step b) takes place by its interaction with electromagnetic radiation, either following steps b) and/or c) or parallel to steps b) and/or c).

The free hydroxy groups of the surface in step a) are preferably parts of a nucleoside and/or nucleotide and comprise, for example, one or more nucleoside structural elements of formula (V), which are linked via 3′-5′ or 5′-3′ phosphoric acid ester: 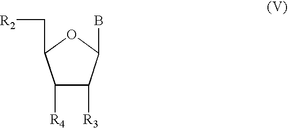
where B can be an H, adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazole-1-yl or 5-amino-4-carbamoylimidazole-1-yl, where in the case where primary amino functions may be present, they could have a permanent protecting group, or thyminyl or uracilyl in the O₄ position could have a permanent protecting group,

R₂ can be H, a phosphoric acid ester residue, a phosphorus amidoester residue, a phosphonic acid ester residue, an H-phosphonate or a suitable hydroxy protecting group,

R₃ can be an H, OH, halogen, acylamino, alkoxy or substituted alkoxy rest with between 1 and 4 C-atoms,

R₄ can be H, a phosphoric acid ester residue, a phosporus amidoester residue, a phosphonic acid ester residue, an H-phosphonate residue or a suitable hydroxy protecting group.

The method according to the invention is preferably performed with compounds of formula (I), where the leaving group L is a suitable chromophoric group that activates the C═Y function by means of lowering the electron density against nucleophilic exchange. Chromophoric groups allow an especially easy quantitative determination by various means of optical spectroscopy methods. Further details with respect to the leaving group L are explained in the foregoing.

Suitable second intermediate protecting groups for the 3′ or 5′ hydroxy function are preferably all protecting groups commonly used by persons skilled in the art, which can be eliminated orthogonally relative to the permanent base protecting groups, but especially photolabile protecting groups. Preferred photolabile second protecting groups are, for example, NPPOC, MeNPOC, MeNNPOC, NPES, NPPS, PyMOC, NVOC, NBOC. The respective reagents are used accordingly for introducing said second protecting groups in the form of their respective alcohols, as for example NPPOH, MeNPOH, MeNNPOH, PyMOH, NVOH, NBOH.

Preferably, the introduction of the second intermediate protecting group is accelerated by commonly used catalysts, such as dimethylaminopyrrolidone, N-methylimidazole, etc.

The object of the present invention is further solved by a method for determining coupling yields in the synthesis of nucleotides, where the method of the invention comprises the following steps:
a) Reacting a free 3′ or 5′ hydroxy group of a selected nucleoside and/or nucleotide with a compound of the general formula (I) 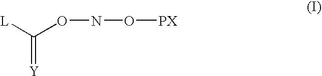
   where L is a common suitable leaving group as discussed in the foregoing, the motif O—PX represents a phosphite amide, a H-phosphonate, a phosphonic acid ester or a phosphotriester, Y═O or S, N is a nucleoside or nucleotide fragment selected from the following general formulae (II) and (III): 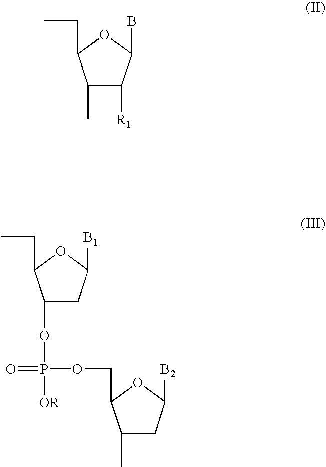
   where the following applies: if the PX-group is in the corresponding terminal nucleosides in the 3′ position in formulae (II) and (III), then the L-C(═Y) unit is in the 5′ position, or if the PX-group is in the 5′ position, then the L-C(═Y) unit is in the 3′ position,
   and B, B₁, B₂ independently can be H, adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazole-1-yl or 5-amino-4-carbamoylimidazole-1-yl, where any primary amino functions that may be present in the case of B, B₁, B₂ could have a permanent protecting group, or thyminyl or uracilyl in the O₄ position could have a permanent protecting group,

R can be H, an alkyl, cycloalkyl, aryl, aralkyl, cyanoalkyl, haloalkyl group,

R₁ can be H, OH, halogen, acylamino, alkoxy or substituted alkoxy with between 1 and 4 C-atoms, or it can form a bicyclic compound via C2′-C4′ cyclization with the ribose unit (LNA)
b) further reaction of the reaction product obtained in step a) with a protecting group reagent and cleavage of the leaving group L, if necessary under catalytic conditions.
c) elimination of the protecting group introduced in step b),
   where the quantitative determination of the amount of the leaving group L eliminated in step b) takes place particularly preferred in the form of its anion L⁻ following steps b) and/or c) or parallel to steps b) and/or c). Preferably, the anion L⁻ is coloured when exposed to visible light, thus interacting with electromagnetic radiation in the UV/VIS range, making it especially easy to determine the quantity of the eliminated anion L⁻, for example by means of UV/VIS and/or fluorescence spectroscopy.

It is especially preferable to automate the above method of the invention.

Preferably, such an automated method is designed as a parallel synthesis for producing an ordered nucleotide library, on a solid surface where the selected oligonucleotides and possibly additional mononucleotides can be selected specifically.

According to another preferred embodiment, the present invention comprises a kit containing some or all of the reagents and/or supplementary agents and/or solvents and/or instructions for performing a method defined in any of the above claims in one spatial unit, where the kit comprises at least one or more selected nucleosides and/or nucleotides.

According to another embodiment the invention comprises the use of the methods of the invention and/or the above mentioned kit for producing oligonucleotides or nucleic acid chips, preferably for an automated and parallelized production of oligonucleotides.

For a better understanding of the invention, the abbreviations and terms used above and below are explained as follows:

DCI 5,6-dicyanoimidazole

DMT dimethoxytrityl-

TsOH toluene sulphonic acid

NPPOC o-nitrophenylpropyloxycarbonyl

PYMOC pyrenylmethyloxycarbonyl

MeNPOC 3,4-methylenedioxy-o-nitrophenylpropyloxycarbonyl

NPS o-nitrophenylethylsulphonyl

NPPS o-nitrophenylpropylsulphonyl

NVOC o-nitroveratryloxycarbonyl

NBOC o-nitrobenzyloxycarbonyl

NPEOC o-nitrophenylethyloxycarbonyl

MeNPPOC 3,4-methylenedioxy-o-nitrophenylpropyloxycarbonyl

The terms nucleoside and nucleotide are used, for example, in accordance with the definitions mentioned in the text book by B. Alberts et al. “Text Book on Molecular Cell Biology” Wiley VCH, Weinheim, N.Y. 1999. The term “nucleotide” for purposes of the present invention includes both oligonucleotides and polynucleotides.

### FIGURES

FIG. 1 shows exemplarily a non-limiting example of a synthesis scheme for performing the method according to the invention.

FIG. 2 shows a further synthesis scheme for performing the method according to the invention.

FIG. 3 shows a biochip (nucleic acid chip) obtained by the method of the invention.

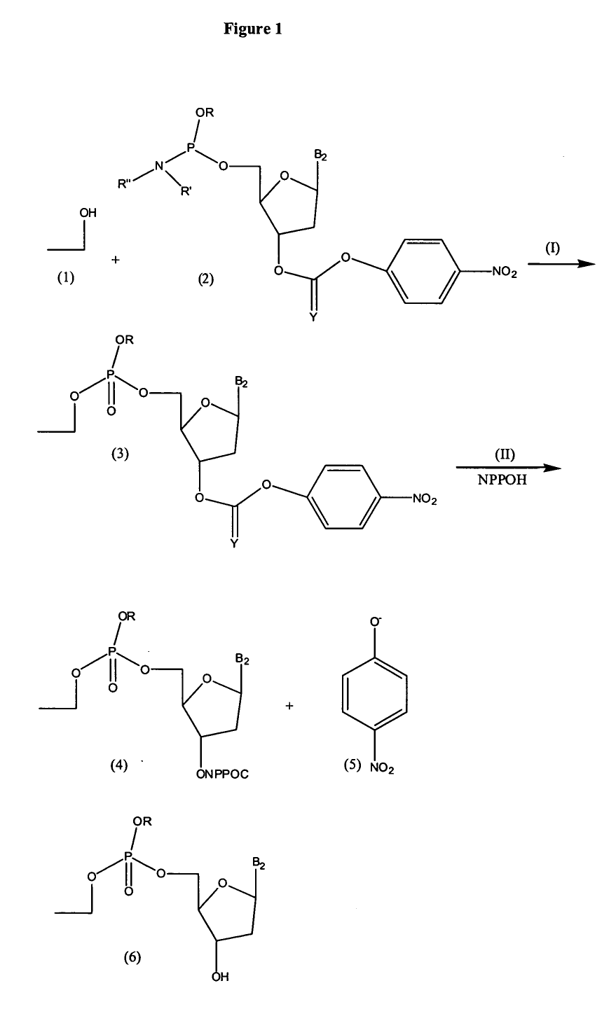

In step (1) of FIG. 1, an OH group is applied to the surface of a freely selectable substrate (also termed as “support”), by means familiar to a person skilled in the art. In another embodiment of the invention the OH group may be part of a nucleoside or a nucleotide, but it is also possible that the surface of the substrate support is already provided with OH groups, for example by using a ceramic, silicon or glass substrate. These substrates also comprise substrate without free hydroxy groups but which are coated with materials having free hydroxy groups. In still another embodiment of the invention, the OH group is a part of an organic or inorganic molecule, for example a silicon molecule, or of long-chain aliphatic or araliphatic alcohols anchored by methods essentially known by an artisan on the substrate surface. In FIG. 1 substrate (1) is a solid support as defined in the foregoing with free hydroxy groups attached to the surface of substrate (1).

In a first reaction step the free hydroxy group(s) is reacted with compound (2), i.e. with a phosphorus amidoester of the above indicated formula. R is H, a branched or unbranched alkyl, preferably a C₁ to C₄ alkyl, cycloalkyl, aryl, aralkyl, cyanoalkyl, most preferably cyanomethyl, cyanoethyl, cyanopropyl, cyanobutyl or a haloalkyl or a heterocyclic residue. R′ and R″ comprise, for example but are not limited to a branched or unbranched alkyl residue with between 1 and 4 C-atoms, for example ethyl or isopropyl, a cycloalkyl or a heterocyclic rest, such as a substituted or non-substituted morpholine rest. The Nitrogen substituents R′ and R″ may be the same or different. If Nitrogen substitutent R′ is different from R″, combinations of the above exemplary groups are preferable. Before performing coupling reaction (I), the phosphorus amidoester must be activated with 1H-tetrazole (TET) or 5,6-dicyanoimidazole (DCI) in acetonitrile. If an H-phosphonate salt is used instead of a phosphorus amidoester, for example, the H-phosphonate salt is activated with pivaloyl chloride or adamantoyl chloride in triethylamine/acetonitrile before reaction with the free hydroxy group. The coupling product may be obtained after oxidation for example with iodine/pyridine of the trivalent phosphor in the form of compound (3).

The intermediate coupling product (3) is reacted with a suitable second protecting group reagent (for example NPPOH with DMAP catalysis), whereby compound (4) is formed. Any other alcohol as mentioned herein is also suited for the purpose of the present invention. It should be noted that any other catalyst instead of DMAP and suitable for this purpose can be used.

The 4-nitrophenolate leaving group (5) can easily be quantitatively determined for example by means of UV/VIS spectroscopy. This allows accurate tracking of whether the coupling reaction is successful, either parallel with or following the coupling step, and allows optimizing of the reaction accordingly. It is also possible to track the leaving group in an online mode, for example by passing the reaction mixture through a photometric cell or placing the substrate in a photometric cell.

In a further step, the NPPOC protecting group of the compound (4) is cleaved via irradiation at a suitable wavelength so that the NPPOC function is converted into the free hydroxy function of compound (6). Compound (6), for example, can then be reused as the parent compound with a free hydroxy group in step a) according to the method of the invention.

As shown exemplarily in FIG. 1, any suitable derivatized hydroxy functions can also be used for performing the method of the invention with nucleosides or nucleotides of the phosphorus amidoester type or H-phosphonates or H-phosphonate salts.

However, nucleosides or polynucleotides that are soluble or bound to a solid phase for example magnetic or non-magnetic beads or other solid phases essentially known by an artisan are also contemplated within the scope of the invention. In these nucleosides or polynucleotides, the terminal 3′ or 5′ hydroxy function is present in the form of a phosphorus amidoester or phosphonic acid ester or H-phosphonate.

It is also possible to use a nucleotide derivative of the following general formula (VI) instead of compound (2) 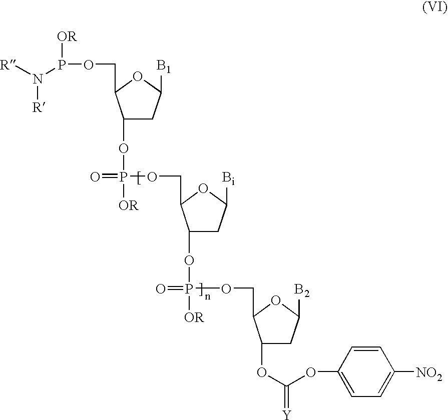
where B, B₁, B₂, Bᵢ independently are H, adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazole-1-yl or 5-amino-4-carbamoylimidazole-1-yl, where any primary amino functions that may be present in the case of B, B₁, B₂, Bᵢ could have a permanent protecting group, or thyminyl or uracilyl in the O₄ position could have a permanent protecting group,

R is H, an alkyl, cycloalkyl, aryl, aralkyl, cyanoalkyl, haloalkyl group,

R₁ is H, OH, halogen, acylamino, alkoxy or substituted alkoxy with between 1 to 4 C-atoms, or it can form a bicyclic compound via C2′-C4′ cyclization with the ribose unit (LNA), Y═O or S, and n=0 or an integer between 1 and 4, preferably 1 and 2 (trimer or tetramer)
or a nucleotide derivative of the general formula (VII) 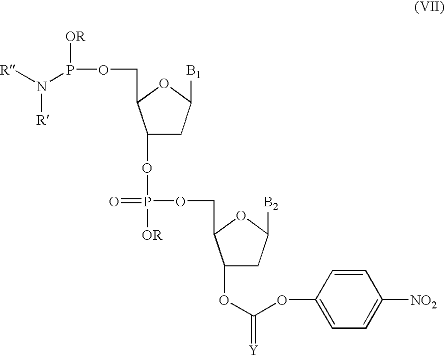
where B₁ und B₂ independently are H, adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazole-1-yl or 5-amino-4-carbamoylimidazole-1-yl, where any primary amino functions that may be present in the case of B₁, B₂ could have a permanent protecting group, or thyminyl or uracilyl in the O₄ position could have a permanent protecting group,

R is H, an alkyl, cycloalkyl, aryl, aralkyl, haloalkyl, cyanoalkyl, group,

R₁ is H, OH, halogen, acylamino, alkoxy or substituted alkoxy with between 1 and 4 C-atoms, or it can produce a bicyclic compound via C2′-C4′ (I) cyclization with the ribose unit (LNA), and Y═O or S.

The meaning of substituents R′ and R″ in formulae (VI) and (VII) corresponds to those as described in formula (II) in FIG. 1.

Of course, with the individual nucleosides or nucleotides it is possible to use nucleosides/nucleotides protected by phosphor derivatives both in 3′ and in 5′ position.

The use of dinucleotides (VII) or oligonucleotides (VI) for the method according to the invention allows a fast and specific formation of longer polynucleotides on derivatized surfaces with higher selectivity and yield because intermediate steps, such as those required in the earlier methods according to the prior art where mononucleotides are used, are now omitted.

Suitable surfaces, comprising substrates and supports include materials, such as films or membranes of polypropylene, nylon, cellulose, cellulose derivatives, for example cellulose acetate, cellulose mixed ester, polyether sulphones, polyamide, polyvinyl chloride, polyvinylidene fluoride, polyester, Teflon or polyethylene.

In addition, the surfaces can also be ceramic materials whose surface has free hydroxy groups. Furthermore, the surfaces can include materials, such as glass, silicon and metals alone or as a coating on other materials.

However, according to another general embodiment of the invention, the use of carrier or coating surfaces with free or protected functional groups is also possible, which have amino, carboxyl, carbonyl, thiol, amide or phosphate groups, for example. Such functional groups can also be linked with the surface via a linker molecule.

Planar carrier surfaces are used for so called “nucleic acid chips”. The term “nucleic acid chips” for purposes of the invention means biomolecules built up on a solid carrier or support. The term “biomolecules” means DNA or RNA, and nucleic acid analogs, such as PNA, LNA or chimerics thereof with DNA, RNA or nucleic acid analogs. The attachment or fixation is achieved via any conventional means essentially known by an artisan.

The oligonucleotide libraries obtained by the method of the invention are preferably used, for example both for hybridization experiments and for certain enzyme reactions (for example DNA ligase, DNA polymerase) on a massive parallel scale.

The methods of the invention are especially well suited for an automated process. Such an automated process is preferably designed as a parallel synthesis for the development of an arrayed nucleotide library.

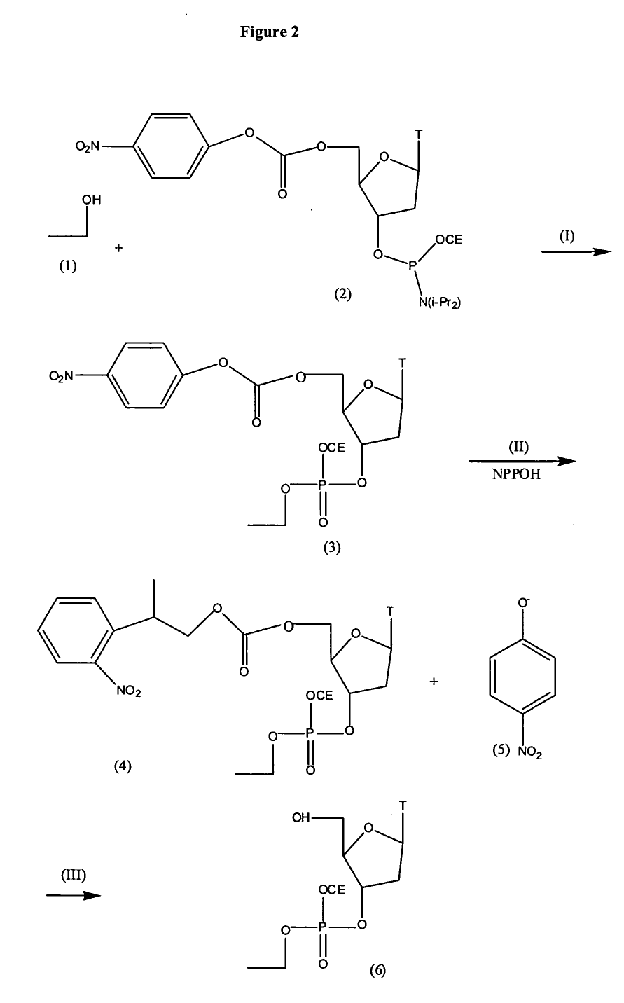

In FIG. 2 free hydroxy groups of a planar surface (1), for example of a biochip, comprising silicones with free hydroxy groups are reacted in step (I) with the thymidine (T) protected nucleoside derivative (2) (CE represents an cyanoethyl group and iPr is an isopropyl group). The reaction is carried out with activation with 1H-tetrazole in acetonitrile. Further, oxidation with iodine and pyridine in water yields compound 3 in very high yields of 96% or more.

The reaction product (3) is reacted with NPPOH under DMAP catalysis conditions in acetonitrile for about 2 minutes. Nucleophilic substitution of the leaving group 4-nitrophenolate (5) takes place. The amount of the free 4-nitrophenolate anion (5) was detected by UV/VIS spectroscopy.

The reaction product 4 is deprotected via usual means under irradiation at a wave length of about 365 nm in DMSO to yield compound 6 with a free hydroxy group which can be used according to the invention, for example as a new substrate with a free hydroxy group.

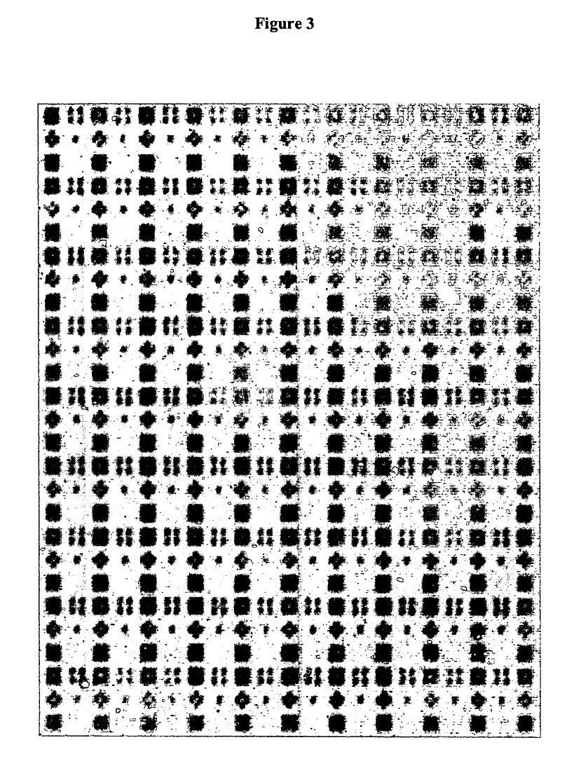

FIG. 3 shows the fluorescence image of a DNA chip obtained according to the invention. The reaction as described in FIG. 2 was carried out. Nucleophilic substitution of the leaving group 4-nitrophenolate first intermediary protecting groups by NPPOH took place followed by deprotection and reaction with fluorescent phosphorus amidite (obtained from Glu Research).

The pattern corresponds to the mirrors commonly used in maskless in situ array synthesis (see e.g. Boguslavsky, J., Drug Discovery and Development, 3, 15-16 (2001))

Fluorescence detection was performed on a Genepix 4000 B fluorescence scanner of Axon Instruments. The fluorescence scanner had a true resolution of 5 μm. As can be seen in FIG. 3, the nucleophilic exchange reaction between the first intermediate protecting group and the second protecting group took place in nearly quantitative yields because the free hydroxy groups of the reaction product reacted with the phosporus amidite.

### EXAMPLES

The following non-limiting examples serve to further explain the present invention:

### Example 1

### 5′-O-(4-Nitrophenyloxycarbonyl)-thymidine

A solution of 4-Nitrophenyl chloroformate (3.6 g, 18 mmol) in Dichloromethane (40 ml) was added dropwise at −15° C. to a stirred solution of Thymidine (5.0 g, 20 mmol) in dry Pyridine (50 ml) and was stirred overnight at 4° C. Methanol was added (0.5 ml), then diluted with Dichloromethane (350 ml), and washed twice with Phosphate buffer solution pH 7.0 (100 ml). The organic phase was dried (Sodium Sulfate), filtered and evaporated. The residue was co-evaporated with Toluene (2×20 ml) and purified by flash column chromatography (fcc) (silica, Ethylacetate). 3.3 g (45%) 5′-O-(4-Nitrophenyloxycarbonyl)-thymidine was obtained as colorless amorphous powder. Crystallisation from Ethylacetate yielded the pure compound of melting point 145-146° C. UV (MeOH, λₘₐₓ nm (logε): 265 (4.28), 212 (4.19). ¹H-NMR (DMSO-d₆, δ in ppm): 11.35 (s, NH); 8.31 (d, 2H, ortho re NO₂); 7.56 (d, 2H, para re NO₂); 7.50 (s, H—C(6)); 6.21 (dd, H—C(1′)); 5.50 (d, HO—C(3′)); 4.40 (m, 3H, H—C(3′) and 2H—C(5′)); 3.99 (m, H—C(4′)); 2.18 (m, 2H—C(2′)); 1.75 (s, Me-C(5)).

### Example 2

### 3′-O-(4-Nitrophenyloxycarbonyl)-thymidine

-Nitrophenylchloroformate (0.5 g, 2.48 mmol) was added to a solution of 5′-O-(4,4′-dimethoxytrityl)-thymidine (1.0 g, 1.83 mmol) in Pyridine/Dichloromethane 10:1 (11 ml). The reaction was stirred overnight at room temperature, quenched with Methanol (0.2 ml), diluted with Dichloromethane (60 ml) and washed twice with Phosphate buffer pH 7.0 (2×30 ml). The organic phase was dried (Sodium Sulfate), filtered and evaporated. The residue was co-evaporated with Toluene (2×20 ml) dissolved in Dichloromethane (15 ml) and added dropwise to Hexanes (150 ml). The precipitate was filtered off, re-dissolved in Dichloromethane (10 ml) and charged with 2%-solution (w/v) of 4-Toluene sulfonic acid in Dichloromethane/Methanol 4:1 (20 ml). After 15 minutes at room temperature Dichloromethane (60 ml) was added and washed with a solution of Sodium Hydrogencarbonate (168 mg) in water (30 ml) und then twice Phosphate buffer pH 7.0 (2×30 ml). The organic phase was dried (Sodium Sulfate), filtered and evaporated. The residue was purified by fcc (silica, Ethylacetate). 0.5 g (67%) of 3′-O-(4-nitrophenyloxycarbonyl)-thymidine was obtained as colorless amorphous powder. UV (MeOH, λₘₐₓ nm (log ε): 265 (4.25), 212 (4.10). ¹H-NMR (DMSO-d₆, δ in ppm): 11.38 (s, NH); 8.32 (d, 2H, ortho zu NO₂); 7.75 (s, H—C(6)); 7.61 (d, 2H, para zu NO₂); 6.23 (dd, H—C(1′)); 5.29 (m, 2H, H—C(3′) and HO—C(5′)); 4.22 (br.s, H—C(4′)); 3.66 (br.s, 2H—C(5′)); 2.37 (m, 2H—C(2′)); 1.78 (s, Me-C(5)).

### Example 3

### 5′-O-(4-Nitrophenyloxycarbonyl)-thymidine-3′-O-[(2-cyanoethyl)-N,N-diisopropyl-phosphoramidite]

5′-O-(4-Nitrophenyloxycarbonyl)-thymidine (1.0 g, 2.45 mmol) and 4,5-Dicyanoimidazole (0.13 g, 1.14 mmol) were dissolved in Dichloromethane (10 ml) and charged with Bis-(diisopropylamino)-2-cyanoethoxyphosphane (1.03 g, 3.43 mmol) in an Argon atmosphere. The reaction was stirred over night at room temperature, diluted with Dichloromethane (100 ml) and washed twice with Phosphate buffer pH 7.0 (2×40 ml). The organic phase was dried (Sodium Sulfate), filtered and evaporated. The residue was purified by fcc (silica, n-Hexane/Acetone 4:1 and 3:2). 0.8 g (54%) 5′-O-(4-Nitrophenyloxycarbonyl)-thymidine-3′-O-[(2-cyanoethyl)-N,N-diisopropylphosphoramidite] was obtained as colorless amorphous powder. UV (MeOH, λₘₐₓ nm (log ε): 265 (4.26), 211 (4.22). ¹H-NMR (DMSO-d₆, δ in ppm): 11.36 (s, NH); 8.31 (d, 2H, ortho re NO₂); 7.53 (d, 3H, para re NO₂ and H—C(6)); 6.20 (dd, H—C(1′)); 4.51 (m, 3H, H—C(3′) and 2H—C(5′)); 4.18 (m, H—C(4′)); 3.74 (m, 2H, POCH₂CH₂); 3.58 (m, 2H, CH(CH₃)₂); 2.79 (t, 2H, CH₂CN); 2.38 (m, 2H—C(2′)); 1.75 (s, Me-C(5)); 1.16 and 1.14(2 s, 12H, iPr). ³¹P-NMR (DMSO-d₆, δ in ppm): 145.46.

### Example 4

### 3′-O-(4-Nitrophenyloxycarbonyl-)thymidine-5′-O-[(2-cyanoethyl)-N,N-diisopropylphos-phoramidite]

3′-O-(4-Nitrophenyloxycarbonyl)-thymidine (1.0 g, 2.45 mmol) and 4,5-Dicyanoimidazol (0.14 g, 1.22 mmol) were dissolved in Dichloromethane (10 ml) and charged with Bis-(Diisopropylamino)-2-cyanoethoxyphosphane (1.1 g, 3.67 mmol) in an Argon atmosphere. The reaction was stirred over night, diluted with Dichloromethane (100 ml) and washed twice with Phosphate buffer pH 7.0 (2×40 ml). The organic phase was dried (Sodium Sulfate), filtered and evaporated. The residue was purified by fcc (silica, n-Hexane/Acetone 4:1 and 3:2). 0.7 g (47%) 3′-O-(4-Nitrophenyloxycarbonyl)-thymidine-5′-O-[(2-cyanoethyl)-N,N-diisopropylphosphoramidite] was obtained as colorless amorphous powder.

UV (MeOH, λₘₐₓ nm (log ε): 265 (4.31), 211 (4.30). ¹H-NMR (DMSO-d₆, δ in ppm): 11.42 (s, NH); 8.33 (d, 2H, ortho re NO₂); 7.60 (m, 3H, para re NO₂ and H—C(6)); 6.23 (m, H—C(1′)); 5.29 (m, H—C(3′)); 4.40 (br.s, H—C(4′)); 3.85 (m, 2H, POCH₂CH₂); 3.77 (m, 2H—C(5′)); 3.55 (m, 2H, CH(CH₃)₂); 2.77 (t, 2H, CH₂CN); 2.54 and 2.38 (2 m, 2H—C(2′)); 1.75 (s, Me-C(5)); 1.13 (m, 12H, iPr). ³¹P-NMR (DMSO-d₆, δ in ppm): 145.50 and 145.44.

### Example 5

### 5′-O-(4,4′-Dimethoxytrityl)-thymidylyl-{3 [O^{P}-(2-cyanoethyl)]→5′}-3′-(4-nitrophenyl-oxycarbonyl-)thymidine

-O-(4,4′-Dimethoxytrityl)-thymidine-3′-O-[(2-cyanoethyl)-N,N-diisopropylphosphoramidite] (0.83 g, 1.11 mmol), 3′-O-(4-nitrophenyloxycarbonyl)-thymidine (0.35 g, 0.85 mmol) and 4,5-Dicyanoimidazol (0.65 g, 5.5 mmol) were dissolved in dry Acetonitrile (8 ml) in an Argon atmosphere and stirred 4 hours at room temperature. A solution of Iodine (0.4 g) in Dichloromethane/water/Pyridine 1:1:3 (v/v/v) (5 ml) was added dropwise until a slight brown coloration remains stable. After 20 minutes Dichloromethane (80 ml) was added and washed twice each with saturated Sodiumthiosulfate in water (2×30 ml) and then Phosphate buffer pH 7.0 (2×30 ml). The organic phase was dried (Sodium Sulfate), filtered and evaporated. The residue was co-evaporated with Toluene (2×15 ml) purified by fcc (silica, Dichloromethane, Dichloromethane/Methanol 50:1 and 20:1). 0.65 g (71%) 5′-O-(4,4′-dimethoxytrityl)-thymidylyl-{3′-[O^{P}-(2-cyanoethyl)]→5′}-3′-O-(4-nitrophenyloxycarbonyl)-thymidine was obtained as colorless amorphous powder. UV (MeOH, λₘₐₓ nm (log ε): 265 (4.45), 236 (4.43), 213 (4.53). ¹H-NMR (DMSO-d₆, δ in ppm): 1.41 and 1.39 (2 s, 2 NH); 8.30 and 8.29 (2 d, 2H, ortho re NO₂); 7.59-6.81 (m, 17H, aromatic and 2H—C(6)); 6.21 (m, 2H—C(1′)); 5.27 and 5.10(2 m, 2H—C(3′)); 4.40-4.11 (m, 6H, 2H—C(4′) and 4H—C(5′)); 3.72 and 3.71 (2 s, 6H, 2 OMe); 3.23(m, 2H, POCH₂CH₂); 2.43 (m, 4H—C(2′)); 1.74 (s, Me-C(6)); 1.41 and 1.39 (2 s, Me-C(6)).

### Example 6

### Thymidylyl-{3′-[O^{P}-(2-cyanoethyl)]→5′}-3′-O-(4-nitrophenyloxycarbonyl)-thymidine

To a solution of 5′-O-(4,4′-dimethoxytrityl)-thymidylyl-{3′-[O^{P}-(2-cyanoethyl)]→5′}-3′-O-(4-nitrophenyloxycarbonyl)-thymidine (0.5 g, 0.47 mmol) in Dichloromethane (2 ml) was added a solution of 2% (w/v) p-Toluene sulfonic acid in Dichloromethane/Methanol 4:1 (4 ml). After 8 minutes at room temperature Dichloromethane (80 ml) was added and washed with a solution of Sodium Hydrogencarbonate (38 mg) in water (30 ml) and then twice Phosphate buffer pH 7.0 (2×30 ml). The organic phase was dried (Sodium Sulfate), filtered and evaporated. The residue was purified by fcc (silica, Dichloromethane, Dichloromethane/Methanol 50:1 and 9:1). 0.3 g (84%) Thymidylyl-{3′-[O^{P}-(2-cyanoethyl)]→5′}-3′-O-(4-nitrophenyloxycarbonyl)-thymidine was obtained as colorless amorphous powder. UV (MeOH, λₘₐₓ nm (log ε): 265 (4.46), 211 (4.41).

### Example 7

### 2′-Deoxy-5′-O-(4,4′-dimethoxytrityl)-N⁴-(4-tert-butylphenyloxyacetyl)-3′-O-(4-nitrophenyl-oxycarbonyl)-cytidine

A solution of 4-Nitrophenylchloroformate (0.36 g, 1.8 mmol) in Dichloromethane (3 ml) was added dropwise to a stirred solution of 2′-Deoxy-5′-O-(4,4′-dimethoxytrityl)-N⁴-(4-tert-butylphenyloxyacetyl)-cytidine (1.0 g, 1.39 mmol) in Pyridine (5 ml) at room temperature. The reaction was stirred over night at room temperature, with Methanol (0.2 ml) quenched and Dichloromethane was added (100 ml) and washed twice with Phosphate buffer pH 7.0 (2×40 ml). The organic phase was dried (Sodium Sulfate), filtered and evaporated. The residue was co-evaporated with Toluene (2×10 ml) and purified by fcc (silica, n-Hexane/Ethylacetate 3:1, 1:1 and 2:3). 1.0 g (81%) of 2′-Deoxy-5′-O-(4,4′-dimethoxytrityl)-N⁴-(4-tert-butylphenyloxyacetyl)-3′-O-(4-nitrophenyloxycarbonyl)-cytidine was obtained as colorless amorphous powder. UV (MeOH, λₘₐₓ nm (log ε): 239 (4.56), 211 (4.70), 300 sh (4.13).

### Example 8

### 2′-Deoxy-N⁴-(4-tert-butylphenyloxyacetyl)-3′-O-(4-nitrophenyloxycarbonyl)-cytidine

To a solution of 2′-Deoxy-5′-O-(4,4′-dimethoxytrityl)-N⁴-(4-tert-butylphenyloxyacetyl)-3′-O-(4-nitrophenyloxycarbonyl)-cytidine (0.7 g, 0.79 mmol) in Dichloromethane (4 ml) was added a solution of 2%—Lösung (w/v) p-Toluene sulfonic acid in Dichloromethane/Methanol 4:1 (8 ml). After 5 minutes stirring at room temperature Dichloromethane (100 ml) was added and washed with a solution of Sodium Hydrogencarbonate (84 mg) in water (50 ml) and then twice with Phosphate buffer pH 7.0 (2×40 ml). The organic phase was dried (Sodium Sulfate), filtered and evaporated. The residue was titurated with n-Hexane, the precipitate filtered and washed with n-Hexane (3×20 ml) und Diethylether (3×15 ml). 0.33 g (72%) 2′-Deoxy-N⁴-(4-tert-butylphenyloxyacetyl)-3′-O-(4-nitrophenyloxycarbonyl)-cytidine was obtained which may be further purified by crystallisation from Ethylacetate/Ethanol 3:1 to a melting point of 196-198° C. UV (MeOH, λₘₐₓ nm (log ε): 249 (4.32), 215 (4.42), 300 sh (4.05).

### Example 9

### 2′-Deoxy-N⁴-(4-tert-butylphenyloxyacetyl)-5′-O-(4-nitrophenyloxycarbonyl)-cytidine

A solution of 4-Nitrophenyl chloroformate (1.74 g, 8.62 mmol) in Dichloromethane (40 ml) was added dropwise to a solution of 2′-deoxy-N⁴-(4-tert-butyloxyacetyl)-cytidine (3.0 g, 7.18 mmol) in Pyridine (30 ml) and Dichloromethane (3 ml) at room temperature. The reaction was stirred over night at room temperature, quenched with Methanol (0.1 ml), diluted with Ethylacetate (200 ml) and washed twice with Phosphate buffer pH 7.0 (2×70 ml). The organic phase was dried (Sodium Sulfate), filtered and evaporated. The residue was co-evaporated with Toluene (2×25 ml) and purified by fcc (silica, Ethylacetate). 1.9 g (45%) 2′-Deoxy-N⁴-(4-tert-butylphenyloxyacetyl)-5′-O-(4-nitrophenyloxycarbonyl)-cytidine was obtained as colorless amorphous powder. UV (MeOH, λₘₐₓ nm (log ε): 248 (4.32), 216 (4.35), 300 sh (4.02).

### Example 10

### 2′-Deoxy-N⁴-(4-tert-butylphenyloxyacetyl)-3′-O-(4-nitrophenyloxycarbonyl)-cytidine-5′-O-[(2-cyanoethyl)-N,N-diisopropylphosphoramidite]

Bis-(diisopropylamino)-2-cyanoethoxyphosphane (0.19 g, 0.64 mmol) was added to a solution of 2′-deoxy-N⁴-(4-tert-butylphenyloxyacetyl)-3′-O-(4-nitrophenyloxycarbonyl)-cytidine (0.25 g, 0.43 mmol) and 4,5-Dicyanoimidazole (25 mg, 0.21 mmol) in Dichloromethane (3 ml) in an Argon atmosphere. After stirring at room temperature for 2 hours Dichloromethane (60 ml) was added and washed three times with Phosphate buffer pH 7.0 (3×30 ml). The organic phase was dried (Sodium Sulfate), filtered and evaporated. The residue was purified by fcc (silica, n-Hexane/Acetone 4:1 and 3:2). 0.17 g (51%) 2′-deoxy-N⁴-(4-tert-butylphenyloxyacetyl)-3′-O-(4-nitrophenyloxycarbonyl)-cytidine-5′-O-[(2-cyanoethyl)-N,N-diisopropyl-phosphoramidite] was obtained as colorless amorphous powder. UV (MeOH, λₘₐₓ nm (log ε): 249 (4.35), 215 (4.42), 300 sh (4.05).

### Example 11

### 2′-Deoxy-N⁴-(4-tert-butylphenyloxyacetyl)-5′-O-(4-nitrophenyloxycarbonyl)-cytidine-3′-O-[(2-cyanoethyl)-N,N-diisopropylphosphoramidite]

Bis-(diisopropylamino)-2-cyanoethoxyphosphane (0.77 g, 2.57 mmol) was added to a solution of 2′-deoxy-N⁴-(4-tert-butylphenyloxyacetyl)-5′-O-(4-nitrophenyloxycarbonyl)-cytidine (1.0 g, 1.71 mmol) and 4,5-Dicyanoimidazole (0.1 g, 0.85 mmol) in Dichloromethane (10 ml) in an Argon atmosphere at room temperature. After stirring for 3 hours Dichloromethane (100 ml) was added and washed three times with Phosphate buffer pH 7.0 (3×30 ml). The organic phase was dried (Sodium Sulfate), filtered and evaporated. The residue was purified by fcc (silica, n-Hexane/Acetone 4:1 and 2:1). 0.63 g (48%) 2′-deoxy-N⁴-(4-tert-butylphenyloxyacetyl)-5′-O-(4-nitrophenyloxycarbonyl)-cytidine-3′-O-[(2-cyanoethyl)-N,N-diisopropylphosphoramidite] was obtained as colorless amorphous powder. UV (MeOH, λₘₐₓ nm (log ε): 248 (4.35), 215 (4.46), 300 sh (4.06).

### Example 12

### 2′-Deoxy-5′-O-(4,4′-dimethoxytrityl)-N-4-(4-tert-butylphenyloxyacetyl)-3′-O-[2-(2-nitrophenyl)propyloxycarbonyl]-cytidine

A solution of 2-(2-Nitrophenyl)-propyl alcohol (266 mg, 1.46 mmol) and 4-(Dimethylamino)-pyridine (22 mg, 0.18 mmol) in Acetonitrile (1 ml) was added to a solution of 2′-Deoxy-5′-O-(4,4′-dimethoxytrityl)-N⁴-(4-tert-butylphenyl-oxyacetyl)-3′-O-(4-nitrophenyloxycarbonyl)-cytidine (130 mg, 0.146 mmol) in Acetonitrile (0.6 ml). After stirring for 2 hours at room temperature the reaction mixture was purified by fcc (silica, n-Hexane/Ethylacetate 2:1 and 2:3). 120 mg (88%) 2′-Deoxy-5′-O-(4,4′-dimethoxtrityl)-N⁴-(4-tert-butylhenyloxyacetyl)-3′-O-[2-(2-nitrophenyl)-propyloxycarbonyl]-cytidine was obtaine as colorless amorphous powder identical to an authentic sample.

### Example 13

### 5′-O-(4,4′-Dimethoxytrityl)thymidylyl-{3′-[O^{P}-(2-cyanoethyl)]→5′}-3′-O-[2-(2-nitrophenyl)propyloxycarbonyl]thymidine

A solution of 2-(2-Nitrophenyl)-propyl alcohol (181 mg, 1 mmol) and 4-(Dimethylamino)-pyridine (12 mg, 0.1 mmol) in Acetonitrile (0.5 ml) was added to a solution of 5′-O-(4,4′-Dimethoxtrityl)-thymidylyl-{3′-[O^{P}-(2-cyanoethyl)]→5′}-3′-O-(4-nitrophenyloxycarbonyl)-thymidine (106 mg, 0.1 mmol) in Acetonitrile (1 ml) at room temperature. After stirring for 30 minutes Dichloromethane (20 ml) was added and washed twice with water (2×20 ml). The organic phase was dried (Sodium Sulfate), filtered and evaporated. The residue was purified by fcc (silica, Dichloromethane and Dichloromethane/Methanol 25:10). 80 mg (72%) of 5′-O-(4,4′-Dimethoxytrityl)-thymidylyl-{3′-[O^{P}(2-cyanoethyl)]-5′}-3′-O-[2-(2-nitrophenyl)-propyloxycarbonyl]-thymidine was obtained as colorless amorphous powder. UV [MeOH, λₘₐₓ nm (log ε)]: 264 (4.36), 236 (4.42), 212 (4.67).

### Example 14

### Reactions of 3′-O-(4-Nitrophenyloxycarbonyl)-thymidine derivatives mit 2-(2-Nitrophenyl)-propyl alcohol

To a mixture of 5′-O-(4,4′-Dimethoxytrityl)-3′-O-(4-nitrophenyloxycarbonyl)-thymidine or 3′-O-(4-Nitrophenyloxycarbonyl)-thymidine respectively (1 equivalent) and 2-(2-Nitrophenyl)-propyl alcohol (app. 10 equivalents) in Acetonitrile was added 4-(Dimethylamino)-pyridine or 1-Methylimidazole (app. 1.1-3.0 equivalents). While stirring at room temperature the reaction turn-over was followed by thin layer chromatography (tic). The 3′-O-[2-(2-nitrophenyl)propyloxycarbonyl]-derivatives were formed quantitatively in a few minutes.

1. A method for the detection of repetitive coupling yields in the synthesis of oligonucleotides characterized in that a leaving group of a first protecting group of a nucleoside or nucleotide hydroxy group is substituted by a second protecting group reagent and wherein the substituted leaving group is detected quantitatively.

2. A method for the detection of repetitive coupling yields in the synthesis of nucleotides according to claim 1, comprising the following steps:
a) reacting a hydroxy group on a surface of free 3′ or 5′ hydroxy group of a selected nucleoside and/or nucleotide with a compound of the general formula (I) 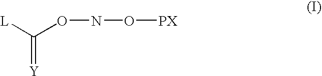
where L is a suitable chromophoric leaving group susceptible to undergo a nucleophilic substitution reaction at the carbon atom of the L-C(═Y)-unit, the motif O—PX represents a phosphor amidite, a H-phosphonate, a phosphonic acid ester or a phosphotriester; Y═O or S; N is a nucleoside or nucleotide fragment selected from the following general formulae (II) or (III): 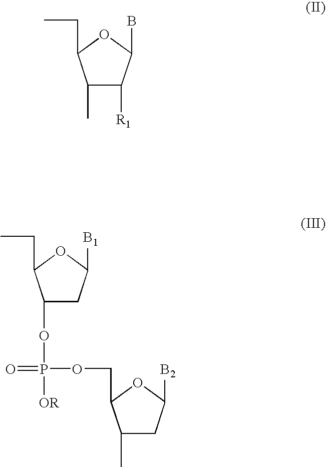
where the following applies: if in the respective terminal nucleosides the PX-group is in the 3′ position in formulae (II) and (III), then the L-C(═Y) unit is in the 5′ position, and if the PX-group is in the 5′ position, then the L-C(═Y) unit is in the 3′ position,
and B, B₁, B₂ independently are H, adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazole-1-yl or 5-amino-4-carbamoylimidazole-1-yl,
R is H, an alkyl, cycloalkyl, aryl, aralkyl, cyanoalkyl, haloalkyl group,
R₁ is H, OH, halogen, acylamino, alkoxy or substituted alkoxy with between 1 and 4 C-atoms, or a bicyclic compound via C2′-C4′ cyclization with a ribose unit (LNA),
b) nucleophilic substitution of the chromophoric leaving group (L), if necessary under catalytic conditions, in the reaction product obtained in step a) by a protecting group reagent suitable for the formation of a second intermediary protecting group,
c) cleavage of the second intermediary protecting group introduced in step b),
whereby the chromophoric leaving group (L) substituted in step b) is quantitatively detected.

3. A method according to claim 2, characterized in that the second protecting group is a photolabile protecting group selected from the following group consisting of: NPPOC, MeNPOC, NVOC, PyMOC, NBOC, NPEOC, MeNPPOC, NPES, or NPPS.

4. A method according to claim 2, wherein the leaving group L is detected in the form of its anion (L⁻) following at least one of steps b) or c) or parallel to at least one of steps b) or c).

5. A method according to claim 4, wherein the detection of the anion (L⁻) takes place by the interaction of the anion (L⁻) with electromagnetic radiation.

6. A method according to claim 5, wherein the interaction is spectroscopically detectable by means of at least one of UV/VIS or fluorescence spectroscopy.

7. A method according to claim 2, characterized in that the steps are carried out in an automated process.

8. A method according to claim 7, characterized in that the automated process is designed as a parallel synthesis for developing a nucleotide library, where the selected nucleosides and/or nucleotides are selected specifically or randomly.

9. A method according to any one of the claims 1 to 8 for the manufacture of oligonucleotides or nucleic acid chips.

10. A method comprising using NPPOH, MeNPOH, PyMOH, MeNPPOH in a method according to any one of the claims 1-8.

11. A kit comprising at least one selected nucleoside or nucleotide for performing the method according to any one of the claims 1-8, and instructions for performing the method according to any one of the claims 1-8 in one spatial unit.

12. A kit according to claim 11, further comprising NPPOH, MeNPOH, PyMOH; MeNPPOH.

13. A nucleoside derivative of the general formula (I) 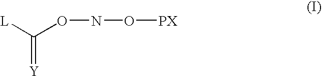
where L is a suitable chromophoric leaving group susceptible to undergo a nucleophilic substitution reaction at the carbon atom of the L-C(═Y)-unit, the motif O—PX represents a phosphor amidite, a phosphonic acid ester, an H-phosphonate or a phosphotriester; Y═O or S; N is a nucleoside or nucleotide fragment selected from the following general formulae (II) and (III): 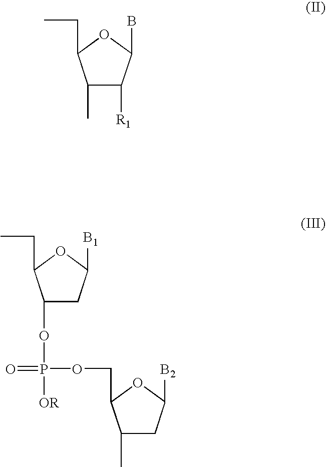
where the following applies: if in the respective terminal nucleosides the PX-group is in the 3′ position in formulae (II) and (III), then the L-C(═Y) unit is in the 5′ position, and if the PX-group is in the 5′ position, then the L-C(═Y) unit is in the 3′ position,
and B, B₁, B₂ independently are H, adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazole-1-yl or 5-amino-4-carbamoylimidazole-1-yl,
R is H, an alkyl, cycloalkyl, aryl, aralkyl, cyanoalkyl, or haloalkyl group,
R₁ is H, OH, halogen, acylamino, alkoxy or substituted alkoxy with between 1 and 4 C-atoms, or it can form a bicyclic compound via C2′-C4′ cyclization with a ribose unit (LNA).

14. A method comprising using a nucleoside derivative according to claim 13 in a method according to any one of claims 1 to 8.

15. A method comprising using a nucleoside derivative according to claim 13 in a kit according to claim 11.

16. A method according to claim 1 where any primary amino functions that may be present in B, B₁, or B₂ have a permanent protecting group, or wherein thyminyl or uracilyl in the O₄ position have a permanent protecting group.

17. A method according to claim 1, wherein the leaving group is detected in the form of its anion.

18. A method according to claim 3, wherein the leaving group L is detected in the form of its anion (L⁻) following at least one of steps b) or c) or parallel to at least one of steps b) or c).

19. A kit according to claim 11, wherein the kit further comprises at least one at least one reagent, supplementary agent or solvent suitable for performing a method according to any one of the claims 1 to 8.

20. A nucleoside derivative according to claim 13 where any primary amino functions that may be present in B, B₁, or B₂ have a permanent protecting group, or wherein thyminyl or uracilyl in the O₄ position have a permanent protecting group.
